(19) United States
(12) Patent Application Publication
(10) Pub. No.: US 2021/0292277 A1
(43) Pub. Date: Sep. 23, 2021

(54) TREATING SARS-COV-2 INFECTED SUBJECTS WITH SMALL MOLECULE COMPOUNDS

(71) Applicant: Anivive Lifesciences, Inc., Long Beach, CA (US)

(72) Inventors: David Bruyette, Long Beach, CA (US); Dylan Balsz, Long Beach, CA (US)

(21) Appl. No.: 17/204,774

(22) Filed: Mar. 17, 2021

Related U.S. Application Data
(60) Provisional application No. 62/991,459, filed on Mar. 18, 2020.
(60) Provisional application No. 63/007,228, filed on Apr. 8, 2020.
(60) Provisional application No. 63/020,138, filed on May 5, 2020.
(60) Provisional application No. 63/024,995, filed on May 14, 2020.

(51) Int. Cl. C07D 207/12; A61K 31/4015; A61K 9/00; A61P 31/14; A61K 45/06; A61K 38/21

(52) U.S. Cl. CPC C07D 207/12; A61K 31/4015; A61K 38/21; A61P 31/14; A61K 45/06; A61K 9/0053

(57) ABSTRACT

The SARS-CoV-2 is highly contagious and has caused coronavirus disease 2019 (COVID-19) outbreaks worldwide. Some embodiments of the present invention relate to a method of ameliorating or treating a subject suffering from a SARS-CoV-2 infection. The method includes administering to the subject an antiviral composition comprising a therapeutically effective amount of a GC molecule or a pharmaceutically acceptable salt thereof.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND

### Field of the Invention

The invention relates generally to biotechnology, especially to virology and medicine. More particularly, disclosed herein are methods of treating coronavirus disease 2019 (COVID-19) caused by severe acute respiratory syndrome coronavirus 2 (SARS-CoV-2) with an antiviral compound, alone or in combination therapy with one or more other agents.

### Description of the Related Art

Coronavirus infections typically result in respiratory and enteric infections affecting both animals and humans. They are considered relatively benign to humans: people around the globe are frequently infected with four human coronaviruses (229E, NL63, OC43, and HKU1) typically leading to an upper respiratory tract infection manifested by common cold symptoms. However, coronaviruses can evolve into a strain that can infect human beings leading to fatal illness, for example, SARS-CoV, MERS-CoV, and the recently identified SARS-CoV-2 (or 2019-nCoV).

Coronaviruses are enveloped positive-stranded RNA viruses that belong to the family Coronaviridae and the order Nidovirales. They possess RNA that is translated directly into one or more polyproteins that are subsequently cleaved by virus proteases into mature or intermediate viral proteins. Those viral proteases are indispensable for virus replication, thus they serve as attractive targets for the design of antiviral compounds. GC376, a chemical compound designed to inhibit the activity of viral protease, has been shown to inhibit viral replication in vitro and in some animal models. See Kim Y, Liu H, Galasiti Kankanamalage A C, Weerasekara S. Hua D H, Groulas W C, el al. (2016) Reversal of the Progression of Fatal Coronavirus Infection in Cats by a Broad-Spectrum Coronavirus Protease Inhibitor. PLoS Pathog 12(3): e1005531: Kim Y, Lovell S, Tiew K C, Mandadapus S R, Allislon K R, Batlaile K P, et al. Broad-spectrum antivirals against 3C or 3C-like proteases of picornaviruses, noroviruses, and coronaviruses. J Virol. 2012; 86 (21):11754-62.

SARS-CoV-2 is highly contagious and has caused coronavirus disease 2019 (COVID-19) outbreaks worldwide. Its infection constitutes an important public health problem. The problem is further exacerbated by the absence of specific anti-SARS-CoV-2 therapeutics or vaccines.

### SUMMARY

Some embodiments of the present invention relate to a method of ameliorating or treating a subject suffering from a SARS-CoV-2 infection, said method comprising administering to said subject an antiviral composition comprising a therapeutically effective amount of a GC molecule or a pharmaceutically acceptable salt thereof.

In another aspect, the antiviral composition further comprises a pharmaceutically acceptable carrier.

In another aspect, the method further comprises administering another therapeutic agent to said subject such as a SARS-CoV-2 RNA-dependent RNA polymerase inhibitor, a cap-dependent endonuclease, protease inhibitors, or a spike inhibitor.

In another aspect, the antiviral composition and the other therapeutic agent are co-administered.

In another aspect, the other therapeutic agent is interferon or SARS-CoV-2 RNA polymerase inhibitor.

In another aspect, both the antiviral composition and the other therapeutic agent are dispersed or dissolved together in a pharmaceutically acceptable carrier.

In another aspect, the administration is an oral administration.

In another aspect, the oral administration is by a tablet comprising the antiviral composition.

In another aspect, the administration is intravenous or subcutaneous administration.

In another aspect, the administration is of a solution comprising the GC molecule or pharmaceutically acceptable salt in a concentration between 1 mg/ml and 500 mg/ml.

In another aspect, the GC molecule or pharmaceutically acceptable salt thereof administered to the subject is in an amount from about 0.1 mg to about 1,000 mg of the GC molecule per kg of body weight of the subject.

In another aspect, this invention provides a method of preventing a subject from suffering from a SARS-CoV-2 infection, said method comprising administering to said subject an antiviral composition comprising a therapeutically effective amount of a GC molecule or a pharmaceutically acceptable salt thereof.

In another aspect, this invention provides a method of preventing or inhibiting replication of SARS-CoV-2 in a cell, said method comprising contacting said cell with an antiviral composition comprising a therapeutically effective amount of a GC molecule or a pharmaceutically acceptable salt thereof.

These and other features, aspects, and advantages of the present invention will become better understood with reference to the following description and appended claims.

### BRIEF DESCRIPTION OF THE DRAWINGS

FIG. 1 is the chemical structure of a GC Molecular Backbone, where R1, R2, R3, or R4 can be substituted for single or multiple deuterated functional groups to improve oral bioavailability and pharmacokinetics of a GC molecule.

FIGS. 2A and 2B show examples of GC molecular analogs containing substitutions of deuterated or ester-containing functional groups. FIG. 2A shows possible substitutions of a GC-376 compound with increased oral bioavailability. FIG. 2B shows possible substitutions of a GC-373 compound with increased oral bioavailability.

FIG. 3 is a line graph which shows the dose response showing the activity of GC376 in SARS-CoV-2 infected cells from a high-throughput drug screening assay.

FIG. 4 is a point graph showing the number of plaque forming units per milliliter from SARS-CoV-2 infected cells with and without treatment with GC376.

FIG. 5A shows the experimental design of a study of GC376 antiviral efficacy in hamsters challenged with SARS-CoV-2 via intranasal infection.

FIG. 5B shows the experimental parameters of the study having the design shown in FIG. 3A.

FIGS. 6A and 6B are line graphs showing body weights over various treatment days in hamsters infected with SARS-CoV-2 and treated with placebo or various doses of GC376. FIG. 6A shows data for six animals evaluated on treatment days 0-3 and two animals evaluated on days 4-8. FIG. 6B shows data for two animals evaluated on treatment days 0-8.

FIG. 7 is a point graph showing body weights for each individual hamster over treatment days 0-8, in hamsters infected with SARS-CoV-2 treated with placebo or various doses of GC376.

FIGS. 8A and 8B show reduction in plaque forming units (PFU) in lung (FIG. 8A) and trachea (FIG. 8B) samples from animals treated with placebo or various doses of GC376.

FIG. 9A shows the experimental design of the antiviral efficacy study of antiviral efficacy of GC376 and REMDESIVIR® against SARS CoV-2 via intranasal challenge in golden Syrian female hamsters.

FIG. 9B shows the procedure for organ harvest and the downstream assays from the assay described in FIG. 9A.

FIG. 10 shows the experimental parameters of the antiviral efficacy study from the assay described in FIG. 9A.

FIGS. 11A and 11B are graphs showing body weights over various treatment days, in hamsters infected with SARS-CoV-2 treated with placebo, GC376 or REMDESIVIR®.

FIG. 12A is a point graph showing reduction in plaque forming units (PFU) in lung samples from animals treated with placebo, GC376, or REMDESIVIR®.

FIG. 12B is a line graph showing the same results as FIG. 12A, but presented in three groups: virus only group at day 4 and day 14, GC376 treatment group at day 4 and day 14, and REMDESIVIR® treatment group at day 4 and day 14.

### DETAILED DESCRIPTION

In the Summary Section above and the Detailed Description Section, and the claims below, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Some embodiments described herein relate to a method of preventing, ameliorating or treating a subject suffering from a SARS-CoV-2 infection. In one embodiment, the method includes administering to the subject an antiviral composition comprising a therapeutically effective amount of a GC molecule or a pharmaceutically acceptable salt thereof. In some embodiments, the GC molecule, such as GC376 is capable of reducing virus titers by more than 15 fold. In some embodiments, administration of the GC molecule is capable of reducing virus titers by more than 10, 11, 12, 13, 14, 15, 16, 17, 18, 19, or 20 fold or more. In some embodiments, the GC molecule is administered prophylactically to prevent a possible exposure to SARS-CoV-2 from developing into COVID-19 disease.

The GC molecules disclosed herein include, but are not limited to, GC373, GC375, GC376, GC523, GC543, GC546, GC551, GC554, GC583, GC587, GC591, GC597, GC772, GC774, GC813, and modifications thereof as discussed below.

The chemical formulas or structures of the above listed GC molecules are as follows:

GC373 and GC375

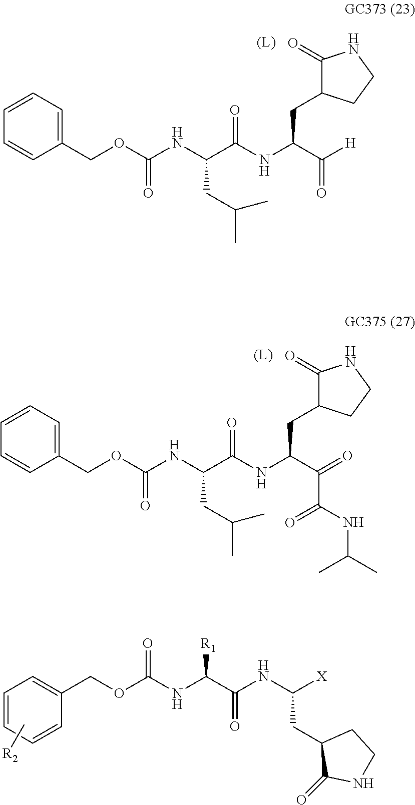

GC376

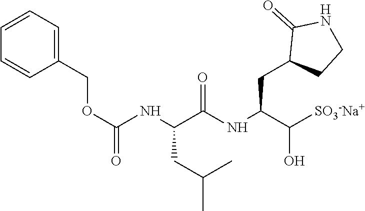

GC543 and GC583

| | | | | EC50 | CC50 |
|---|---|---|---|---|---|
| Comp | R1 | R2 | X | (μM) | (μM) |
| GC543 | Cha | H | CHO | 0.06 | >100 |
| GC583 | Cha | m-Cl | CHO | 0.02 | >100 |

GC546, GC551, and GC554

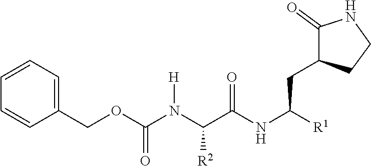

| Compounds | R¹ | R² |
|---|---|---|
| GC546 | CHO | Benzyl (Phe) |
| GC551 | CH(OH) SO₃Na | Cyclohexylmethyl (Cha) |
| GC554 | CH(OH) SO₃Na | Benzyl (Phe) |

GC813

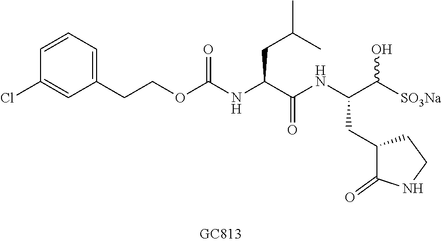

GC523, GC587, GC591, GC597, GC772, and GC774

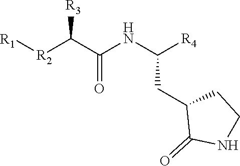

| Compound | R₁ (Cap) | R₂ | R₃ | R₄ (war head) |
|---|---|---|---|---|
| GC523 | | | | (C═O)CONH cyclohexyl |
| GC587 | | | | CH(OH)SO₃Na |
| GC591 | | | | (C═O)CONH Cyclopropyl |
| GC597 | | | Leu | CHO |
| GC772 | m-Cl(C₆H₄) | SO₂NH | Cha | CHO |
| GC774 | p-Cl(C₆H₄) | SO₂NH | | |

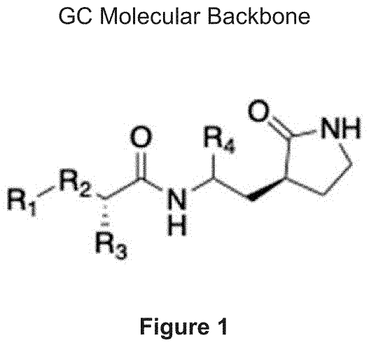

Some GC molecules have the molecular backbone shown in FIG. 1, where R1, R2, R3, or R4 can be substituted for single or multiple deuterated functional groups to improve oral bioavailability and pharmacokinetics.

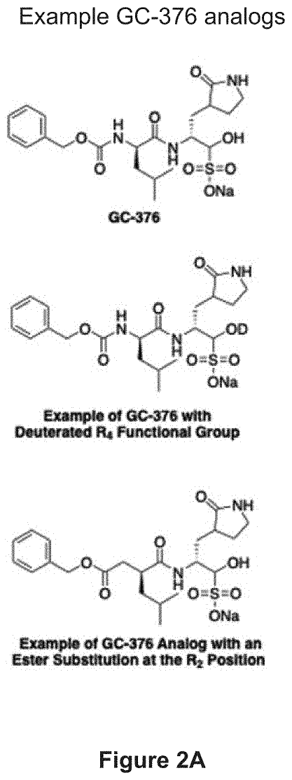

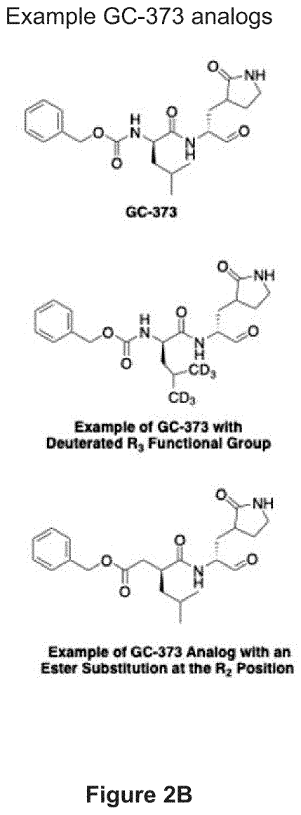

FIGS. 2A and 2B are some examples of GC molecules that include molecular analogs containing substitutions of deuterated or ester-containing functional groups. FIG. 2A shows possible substitutions of a GC-376 molecule with increased oral bioavailability. FIG. 2B shows possible substitutions of a GC-373 molecule with increased oral bioavailability.

As used herein, a “therapeutically effective amount” refers to a sufficient amount of a GC molecule to treat SARS-CoV-2 infected subjects, at a reasonable benefit/risk ratio applicable to any medical treatment. It will be understood, however, that the total daily usage of the GC molecule will be decided by the attending physician within the scope of sound medical judgment. The specific therapeutically effective dose level for any particular subject will depend upon a variety of factors including the disorder being treated and the severity of the disorder; the specific composition employed, the age, body weight, general health, sex and diet of the subject; the time of administration, route of administration, and rate of excretion of the GC molecule employed; the duration of the treatment; drugs used in combination or coincidental with the GC molecule; and like factors well known in the medical arts. For example, it is well known within the skill of the art to start doses of the compound at levels lower than those required to achieve the desired therapeutic effect and to gradually increase the dosage until the desired effect is achieved. In addition, a “therapeutically effective amount” is the amount that will elicit the biological or medical response of a tissue, system, or subject that is being sought by a researcher or clinician, and in particular elicit some desired therapeutic or prophylactic effect as against the viral infection by preventing and/or inhibiting 3C or 3CL protease activity and/or viral replication.

One of skill in the art recognizes that an amount may be considered therapeutically “effective” even if the condition is not totally eradicated or prevented, but it or its symptoms and/or effects are improved or alleviated partially in the subject. Various indicators for determining the effectiveness of a method for treating a virus infection are known to those skilled in the art. Examples of suitable indicators include, but are not limited to, a reduction in viral load, a reduction in viral replication, a reduction in time to seroconversion (virus undetectable in patient serum), an increase in the rate of sustained viral response to therapy, a reduction of morbidity or mortality in clinical outcomes, and/or other indicator of disease response.

In some embodiments, the composition comprises from about 5% to about 10%, 20%, 30%, 40%, 50%, 60%, 70%, 80%, 90%, or 95% by weight of the GC molecule, and preferably from about 30% to about 90% by weight of the GC molecule, based upon the total weight of the composition taken as 100% by weight.

Other ingredients may be included in the antiviral composition, such as other active agents, preservatives, buffering agents, salts, a pharmaceutically acceptable carrier, or other pharmaceutically acceptable ingredients.

The subject suffering from a SARS-CoV-2 infection can be humans and other animals.

In some embodiments, the method further comprises administering another therapeutic agent to the subject. In some embodiments, the therapeutic agents can be, but are not limited to, SARS-CoV-2 RNA-dependent RNA polymerase inhibitor, a cap-dependent endonuclease, protease inhibitors, a spike inhibitor, or interferons. Examples of suitable RNA polymerase inhibitors include, but are not limited to, REMDESIVIR® (development code GS-5734) by Gilead Sciences. In some embodiments, the other therapeutic agent is interferon, such as a pegylated interferon. Examples of suitable interferons include, but are not limited to, Pegylated interferon-alpha-2a (brand name PEGASYS®), Pegylated interferon-alpha-2b (brand name PEG-INTRON®), interferon alfacon-1 (brand name INFERGEN®), pegylated interferon lambda and/or a combination thereof. In some embodiments, the antiviral composition and the other therapeutic agent are co-administered. In one embodiment, the order of administering the antiviral compound and the other therapeutic agents can be in any order.

A potential advantage of utilizing a GC molecule, or a pharmaceutically acceptable salt thereof, in combination with one or more additional agent(s) is that the use of two or more compounds having different mechanism of actions can create a higher barrier to the development of resistant SARS-CoV-2 viral strains compared to the barrier when a compound is administered as monotherapy.

In some embodiments, both the antiviral composition and the other therapeutic agent are dispersed or dissolved together in a pharmaceutically acceptable carrier.

In some embodiments, the administration of the antiviral composition is of a solution comprising a GC molecule or pharmaceutically acceptable salt in a concentration between 1 mg/ml and 500 mg/ml.

The dosage may range broadly, depending upon the desired effects and the therapeutic indication. The daily dosage regimen for an adult human patient may be, for example, an oral dose of between 0.01 mg and 3000 mg of a GC molecule, preferably between 1 mg and 700 mg, e.g. 5 to 200 mg, or between about 0.1 mg and about 1,000 mg of the GC molecule per kg of body weight of the subject. The dosage may be a single one or a series of two or more given in the course of one or more days, as is needed by the subject. In some embodiments, the compounds are administered for a period of continuous therapy, for example for a week or more, or for months or years. In some embodiments, the GC molecule, or a pharmaceutically acceptable salt thereof, can be administered less frequently compared to the frequency of administration of an agent within the standard of care. In some embodiments, the GC molecule, or a pharmaceutically acceptable salt thereof, can be administered one time per day. For example, the GC molecule, or a pharmaceutically acceptable salt thereof, can be administered one time per day to a subject suffering from a SARS-CoV-2 infection. In some embodiments, the total time of the treatment regime with the GC molecule, or a pharmaceutically acceptable salt thereof, can be less compared to the total time of the treatment regime with the standard of care.

In instances where human dosages for a GC molecule have been established for at least some condition, those same dosages may be used, or dosages that are between about 0.1% and 500%, more preferably between about 25% and 250% of the established human dosage. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the above-stated, preferred dosage range in order to effectively and aggressively treat particularly aggressive diseases or infections.

Dosage amount and interval may be adjusted individually to provide plasma levels of the active moiety that are sufficient to maintain the modulating effects, or minimal effective concentration (MEC). Dosages necessary to achieve the MEC will depend on individual characteristics and route of administration. However, HPLC assays or bioassays can be used to determine plasma concentrations. Dosage intervals can also be determined using MEC value. Compositions should be administered using a regimen that maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Antiviral compositions disclosed herein can be evaluated for efficacy and toxicity using known methods. For example, the toxicology of a particular antiviral composition may be established by determining in vitro toxicity towards a cell line, such as a mammalian, and preferably human, cell line. The results of such studies are often predictive of toxicity in animals, such as mammals, or more specifically, humans. Alternatively, the toxicity of particular antiviral composition in an animal model, such as mice, rats, rabbits, or monkeys, may be determined using known methods. The efficacy of a particular antiviral composition may be established using several recognized methods, such as in vitro methods, animal models, or human clinical trials. When selecting a model to determine efficacy, the skilled artisan can be guided by the state of the art to choose an appropriate model, dose, route of administration and/or regime.

In some embodiments, the subject is afflicted with or suffering from a condition (e.g., infection, disease, or disorder) before the compounds are administered, wherein methods described herein are useful for treating the condition and/or ameliorating the effects of the condition. In other embodiments, the subject is free of a given condition before administering the compound, wherein the methods described herein are useful for preventing the occurrence or incidence of the condition and/or preventing the effects of the condition, as described above. Some embodiments described herein relate to a method of preventing a subject from suffering from a SARS-CoV-2 infection, said method comprising administering to said subject an antiviral composition comprising a therapeutically effective amount of a GC molecule or a pharmaceutically acceptable salt thereof.

The disclosed embodiments are suitable for various routes of administration, depending upon the particular carrier and other ingredients used. For example, the prophylactic and/or therapeutic compounds or compositions can be injected intramuscularly, subcutaneously, intradermally, or intravenously. In some embodiments, the administration is of a solution comprising a GC molecule or pharmaceutically acceptable salt in a concentration between 1 mg/ml and 500 mg/ml. The antiviral composition can also be administered via mucosa, such as intranasally or orally. In some embodiments, the oral administration is by a tablet comprising the antiviral composition. The compounds or compositions can also be administered through the skin via a transdermal patch.

In some embodiments, this invention provides a method of preventing or inhibiting replication of SARS-CoV-2 in a cell. In one embodiment, the method includes contacting the cell with an antiviral composition comprising a therapeutically effective amount of a GC molecule or a pharmaceutically acceptable salt thereof. Some embodiments relate to use of a GC molecule in the production of a medicament for the treatment of COVID-19 or a SARS-CoV-2 infection in a subject.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluenesulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine and lysine.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans. In some embodiments, the subject is human.

As used herein, the terms “treating,” “treatment,” “therapeutic,” or “therapy” do not necessarily mean total cure or abolition of the disease or condition. Any alleviation of any undesired signs or symptoms of a disease or condition, to any extent can be considered treatment and/or therapy. Furthermore, treatment may include acts that may worsen the patient's overall feeling of well-being or appearance.

As used herein, the term “ED₅₀” refers to the dose that produces the desired effect in 50% of the population, or median effective dose. The term “ID₅₀” refers the concentration of antiviral drug required to reduce the virus-specific DNA or RNA by 50% compared with the untreated virus controls.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It is understood that the methods and combinations described herein include crystalline forms (also known as polymorphs, which include the different crystal packing arrangements of the same elemental composition of a compound), amorphous phases, salts, solvates and hydrates. In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

### Antiviral Compositions

Some embodiments described herein relates to an antiviral composition, which can include an effective amount of a GC molecule, or a pharmaceutically acceptable salt thereof and a pharmaceutically acceptable carrier, diluent, excipient or combination thereof.

The term “antiviral composition” refers to a mixture of a GC molecule with other chemical components, such as diluents or carriers. The antiviral composition facilitates administration of the compound to an organism. Antiviral compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid and salicylic acid. Antiviral compositions will generally be tailored to the specific intended route of administration. An antiviral composition is suitable for human and/or veterinary applications.

As used herein, a “carrier” refers to a compound that facilitates the incorporation of a compound into cells or tissues. For example, without limitation, dimethyl sulfoxide (DMSO), Ethanol (EtOH), or PEG400 is a commonly utilized carrier that facilitates the uptake of many organic compounds into cells or tissues of a subject.

As used herein, a “diluent” refers to an ingredient in an antiviral composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to an antiviral composition to provide, without limitation, bulk, consistency, stability, binding ability, lubrication, disintegrating ability etc., to the composition. A “diluent” is a type of excipient.

The antiviral compositions described herein can be administered to a human patient per se, or in antiviral compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The antiviral compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections.

One may also administer the compound in a local rather than systemic manner, for example, via injection of the compound directly into the infected area, often in a depot or sustained release formulation. Furthermore, one may administer the compound in a targeted drug delivery system, for example, in a liposome coated with a tissue-specific antibody. The liposomes will be targeted to and taken up selectively by the organ.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a GC molecule formulated in a compatible pharmaceutical carrier may also be prepared, placed in an appropriate container, and labeled for treatment of an indicated condition.

### Example 1: Activity of GC376

Activity of GC376 against SARS-CoV-2 protease in a fluorescence resonance energy transfer (FRET) assay

Stock solutions (10 mM) of the substrates and GC376 were prepared in dimethyl sulfoxide (DMSO) and diluted in assay buffer. The assay buffer comprised 20 mM HEPES buffer containing NaCl, 0.4 mM EDTA, glycerol and dithiothreitol (DTT) at pH 6. The SARS-CoV-2 3CLPro protease was mixed with serial dilutions of GC376 or with DMSO in 25 μl of assay buffer and incubated at 37° C. for 30 min, followed by the addition of 25 μl of assay buffer containing substrate. Fluorescence readings were obtained using an excitation wavelength of 360 nm and an emission wavelength of 460 nm on a fluorescence microplate reader 1 h following the addition of substrate. Relative fluorescence units (RFU) were determined by subtracting background values (substrate containing well without protease) from the raw fluorescence values.

The FRET results showed that the IC₅₀ of GC376 against SARS-CoV-2 3CLPro protease is 2.4 μM.

### Activity of GC376 in a High-Throughput Screen of Animal and Human Coronaviruses

A high-throughput drug screening assay was used to look for compounds that worked against dozens of viral proteases at the same time. In this assay, yeast cells were used to express the individual viral protease. Using a set of >50 viral proteases a single assay was able to test GC376 against 17 coronavirus proteases from human and animal coronaviruses. GC376 was shown to have activity across all coronaviruses tested, including SARS-CoV-2.

### Activity of GC376 Against SARS-CoV-2

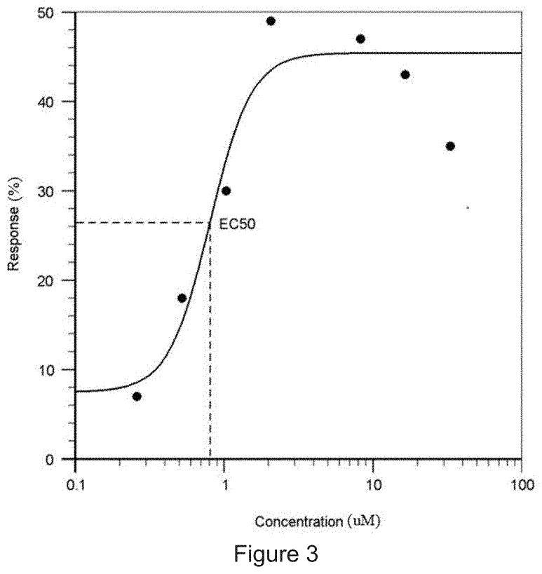

A high-throughput drug screening assay was used to analyze the activity of GC376 in SARS-CoV-2 infected cells. The dose response data was input to make the graph shown in FIG. 3 and the data shows that the EC50 of GC376 against SARS-CoV-2 is about 0.81 μM.

### Effect of GC376 on Suppressing SARS-CoV-2 Activity

COVID-19/SARS-CoV-2 virus solutions with or without GC376 were added to plates with cells at about 90-100% confluence. The experiment was done in triplicate. After adding agarose and growth media, the plaque-forming units (PFU) were then counted on each plate to determine the in vitro effect of GC376 on SARS CoV-2 viral growth.

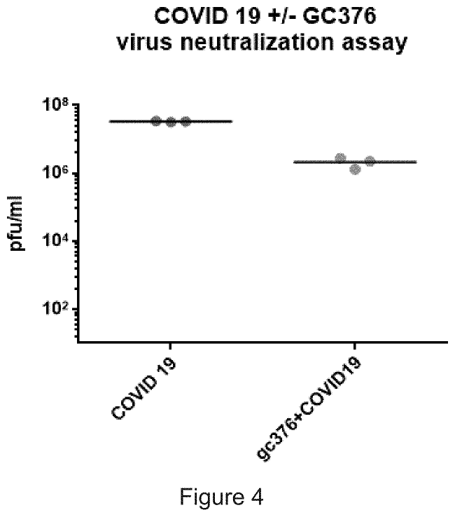

The PFU/ml for the virus solution with 5 μM GC376 averaged 2.1×10⁶ pfu/ml from three replicate plates (2.75×10⁶, 2.25×10⁶, 1.30×10⁶ for each plate, respectively). For the virus solution without GC376, the average pfu/ml was 3.33×10⁷ from three replicate plates (3.23×10⁷, 3.43×10⁷, 3.33×10⁷ for each plate, respectively). The results can be seen in the graph of FIG. 4. The data show that 5 μM GC376 lowered the COVID-19/SARS-CoV-2 virus titer by approximately 15 fold (3.33×10⁷/2.1×10⁶), demonstrating that GC376 has significantly suppressed the activity of COVID-19/SARS-CoV-2 viral growth in vitro.

### Example 2

This example shows GC376 antiviral efficacy in hamsters challenged with SARS-CoV-2 via intranasal infection.

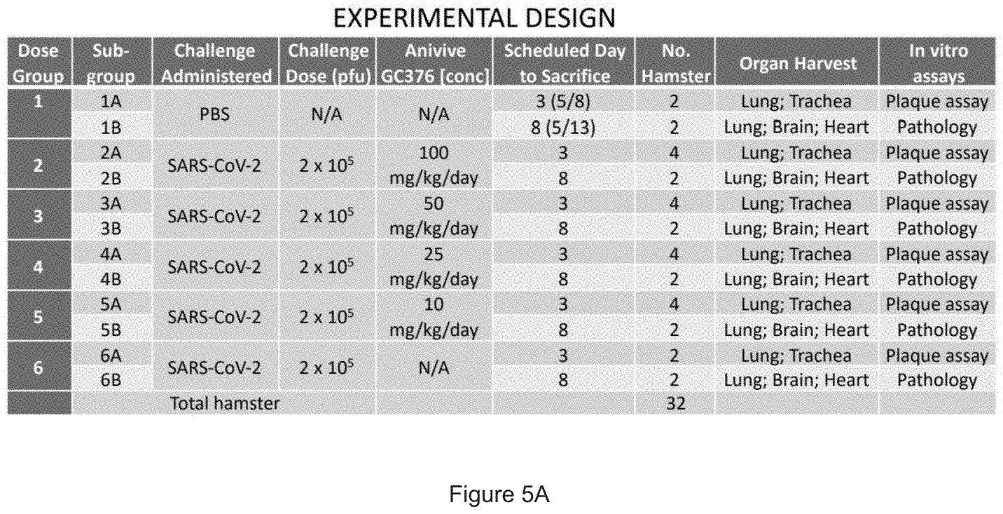

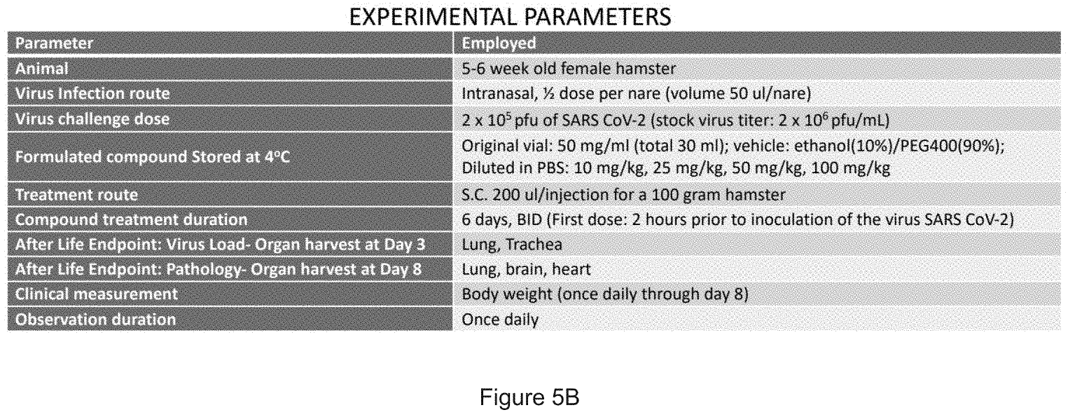

Briefly, 5 to 6-week-old female hamsters were intranasally introduced with 2×10⁵ pfu SARS-CoV-2 virus or PBS, then the hamsters were S.C injected with GC376 solutions at various concentration. The hamsters were sacrificed and various tissues were collected for analysis. The detailed experimental design is shown in FIG. 5A and the experimental parameters are shown in FIG. 5B.

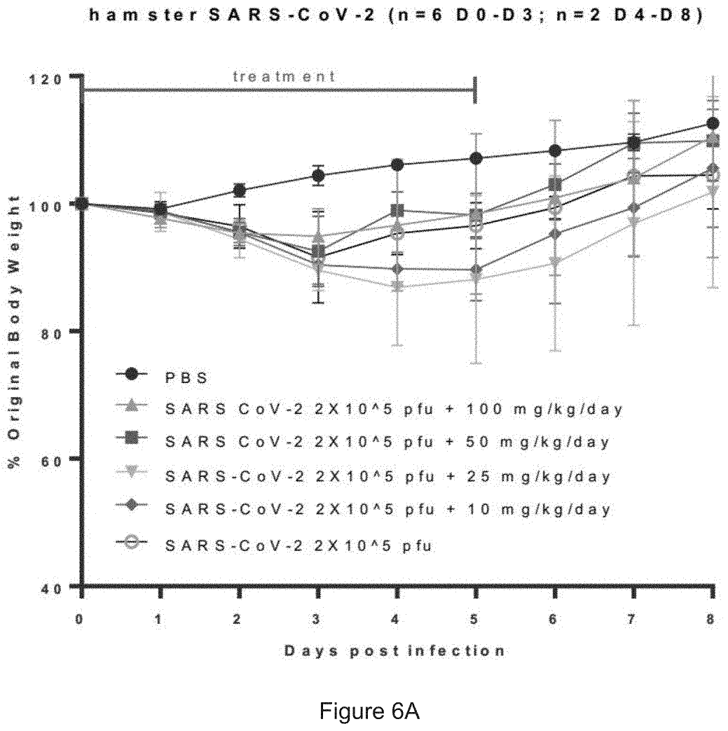

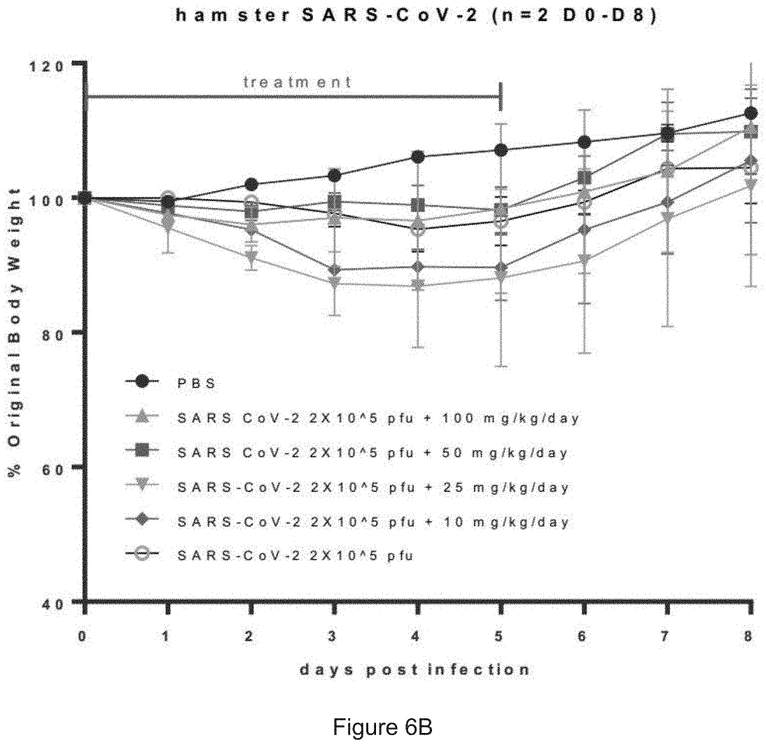

FIGS. 6A and 6B show percent body weight changes over various treatment days in hamsters infected with SARS-CoV-2 treated with placebo or various doses of GC376. As shown, animals infected with SARS-CoV-2 initially dropped in weight over the five days of treatment, but then gained back their weight to 100% of their original weight by the eighth day following initiation of the experiment. Animals treated at 10 mg/kg/day and 25 mg/kg/day appeared to lose more weight initially, but by the eighth day following initiation of the experiment had a body weight that was similar to the control animals who received SARS-CoV-2, but no dose of GC376. The data show that there is a dose dependent effect of GC376 on reducing weight loss and accelerating weight gain back towards baseline.

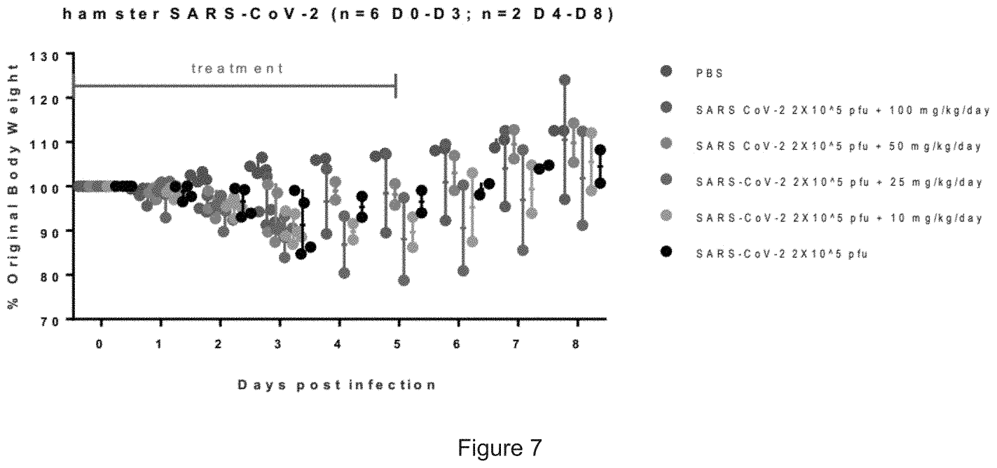

FIG. 7 shows body weights for each individual hamster over treatment days 0-8, in hamsters infected with SARS-CoV-2 treated with placebo or various doses of GC376. As shown, the hamsters receiving the higher doses of GC376 appeared to have a wider range of weight changes than hamsters receiving lower doses of GC376. In addition, the data shows that no matter what dose of GC376 was given to the animals, there was a trend show for each animal to regain its normal body weight following infection with SARS-CoV-2.

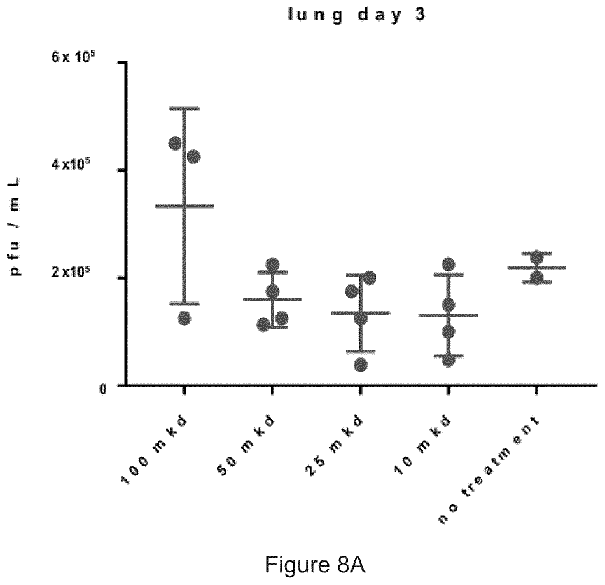

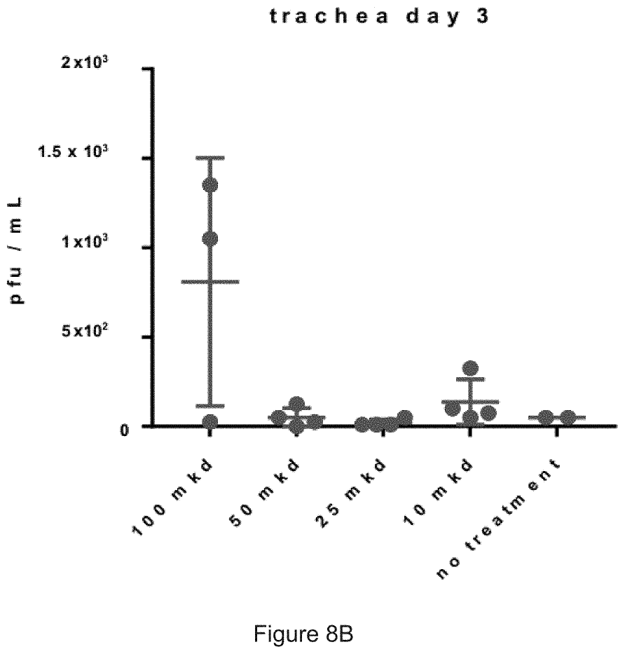

FIGS. 8A and 8B show reduction in SARS-CoV-2 plaque forming units (PFU) in lung (FIG. 8A) and trachea (FIG. 8B) samples from animals treated with placebo or various doses of GC376. As shown the animals treated with 10, 25 and 50 mg/kg/day of GC376 had on average fewer PFU in their lungs than control animals who did not receive any treatment. Animals having no treatment had more than 2×10⁵ PFU/ml whereas animals treated with 10, 25 or 50 mg/kg/day had about 1.2×10⁵ PFU/ml to 1.5×10⁵ PFU/ml on average, with some animals in the 10 and 25 mg/kg/day treatment groups showing substantial reductions in lung PFU/ml of between 0.2×10⁵ PFU/ml to 0.4×10⁵ PFU/ml. One animal in the cohort which received 100 mg/kg/day of GC376 had fewer PFUs in their lungs than the control animal, although two animals had more PFUs than the control. Referring to FIG. 8B, it can be seen that most of the animals did not show PFUs from samples taken from the trachea, although two samples from the 100 mg/kg/day group did show noticeable PFU formation from trachea samples.

### Example 3

This example shows results of antiviral efficacy of GC376 in comparison to the antiviral drug REMDESIVIR® against SARS CoV-2 via intranasal challenge in golden Syrian female hamsters.

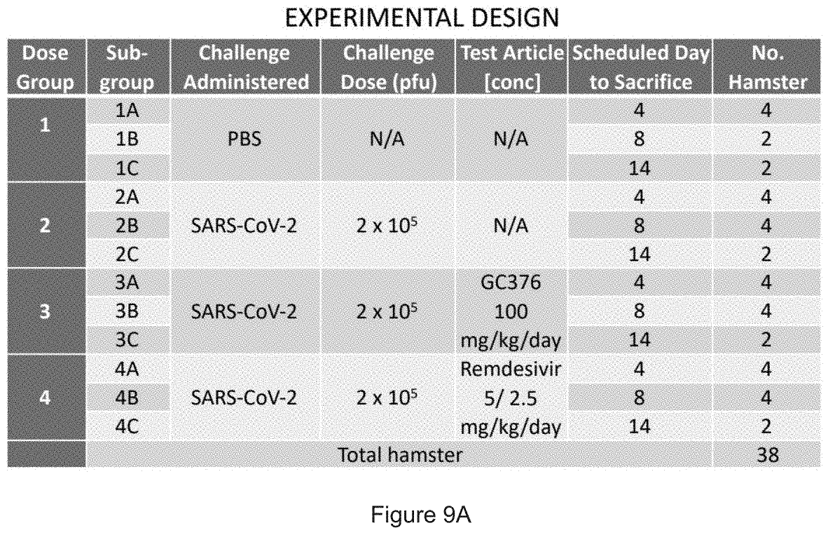

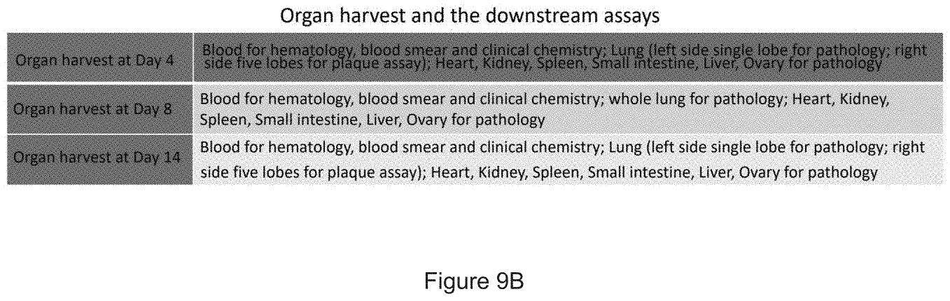

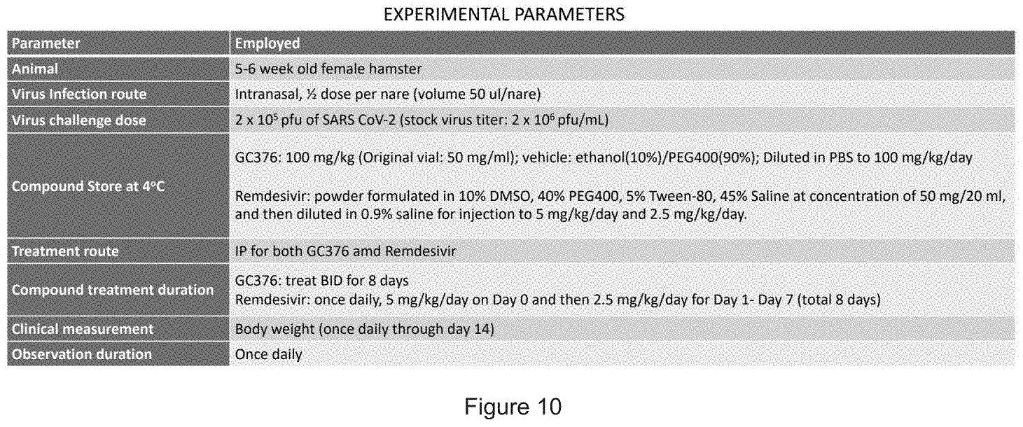

Briefly, 5 to 6-week-old female hamsters were intranasally introduced with 2×10⁵ pfu SARS-CoV-2 virus or PBS, then the hamsters were treated with GC376 or a REMDESIVIR® solution at various concentration through IP injection for eight days. The animals were sacrificed at days 4, 8, or 14 following initiation of the experiment and the various tissues were then collected for analysis. Detailed experimental design and experimental parameters can be found in FIG. 9A, FIG. 9B, and FIG. 10.

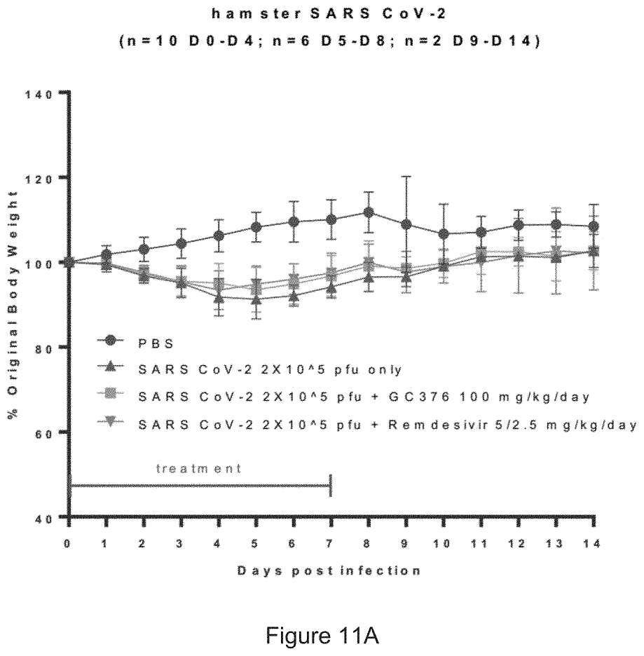

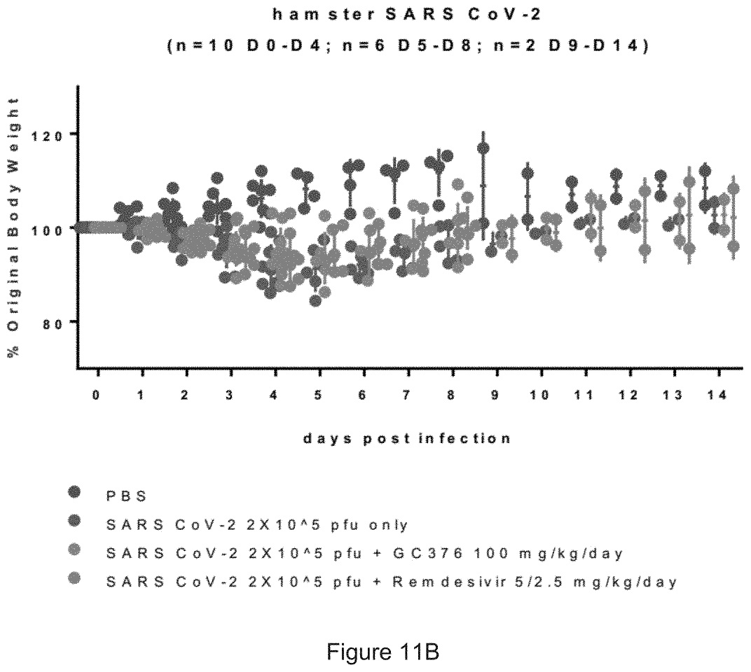

FIGS. 11A and 11B show body weights over various treatment days. As shown, hamsters infected with SARS-CoV-2 and treated with GC376 or REMDESIVIR® maintained similar body weights to the control animals who received SARS-CoV-2 alone.

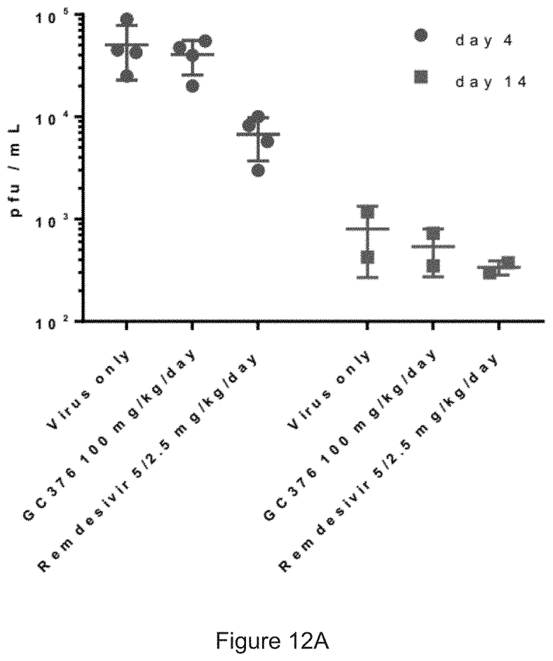

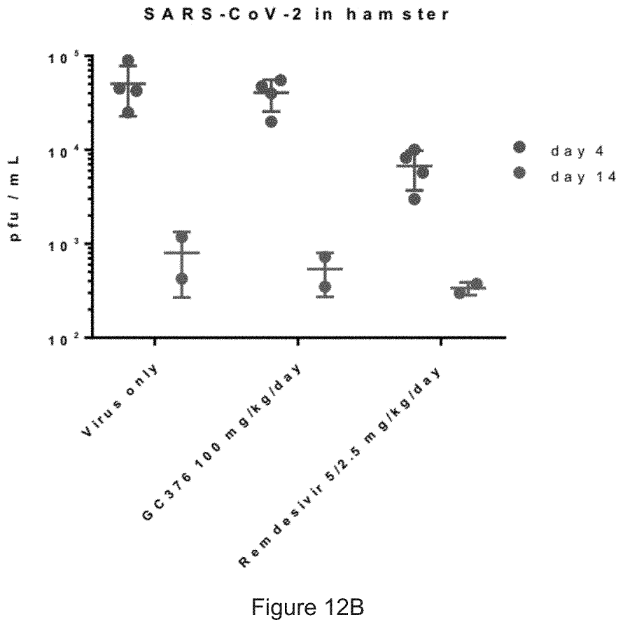

FIG. 12A shows reduction in SARS-CoV-2 plaque forming units (PFU) in lung samples from animals treated with placebo, GC376, or REMDESIVIR®. FIG. 12B shows the same result but presents in three groups: virus only group at day 4 and day 14, GC376 treatment group at day 4 and day 14, and REMDESIVIR® treatment group at day 4 and day 14 The data shows that treatment with GC376 or REMDESIVIR® results in a reduction of PFU at days 4 and 14.

### Example 4: Treatment of a Patient Suffering from COVID-19

A human patient is tested and found to be positive for having a SARS-CoV-2 viral infection. The patient is given intravenous, subcutaneous, or oral administration of a composition comprising GC376 daily for 21 days. After 21 days of treatment, the patient is found to have recovered from the SARS-CoV-2 infection.

### Example 5: Prophylactic Treatment of a Patient Susceptible to COVID-19

A human patient at risk for being exposed to the SARS-CoV-2 virus is prophylactically administered GC376. The patient is given intravenous, subcutaneous, or oral administration of a composition comprising GC376 daily for 21 days, beginning with one week prior to the possible exposure to SARS-CoV-2. After 21 days of treatment, the patient is found to not have developed COVID-19 or a SARS-CoV-2 infection following the prophylactic treatment.

What is claimed is:

1. A method of treating a subject suffering from a SARS-CoV-2 infection, said method comprising administering to said subject an antiviral composition comprising a therapeutically effective amount of a GC molecule or a pharmaceutically acceptable salt thereof.

2. The method of claim 1 wherein the GC molecule is selected from the group consisting of: GC373, GC375, GC376, GC523, GC543, GC546, GC551, GC554, GC583, GC587, GC591, GC597, GC772, GC774, and GC813.

3. The method of claim 1, wherein the GC molecule is GC376.

4. The method of claim 1, further comprising administering another therapeutic agent to said subject.

5. The method of claim 4, wherein the other therapeutic agent is an interferon, a SARS-CoV-2 RNA-dependent RNA polymerase inhibitor, a cap-dependent endonuclease, a protease inhibitor, or a spike inhibitor.

6. The method of claim 4, wherein both the antiviral composition and the other therapeutic agent are dispersed or dissolved together in a pharmaceutically acceptable carrier.

7. The method of claim 1, wherein the administration is an oral administration.

8. The method of claim 7, wherein the oral administration is by a tablet comprising the antiviral composition.

9. The method of claim 1, wherein the administration is intravenous or subcutaneous administration.

10. The method of claim 9, wherein the administration is of a solution comprising the GC molecule or pharmaceutically acceptable salt in a concentration between 1 mg/ml and 500 mg/ml.

11. The method of claim 1, wherein the subject is a human subject.

12. The method of claim 1, wherein the GC molecule or pharmaceutically acceptable salt thereof administered to the subject is in an amount from about 0.1 mg to about 1,000 mg of the GC molecule per kg of body weight of the subject.

13. A method of preventing a subject from suffering from a SARS-CoV-2 infection, said method comprising administering to said subject at risk for being exposed to SARS-CoV-2 an antiviral composition comprising a therapeutically effective amount of a GC molecule or a pharmaceutically acceptable salt thereof.

14. The method of claim 13 wherein the GC molecule is selected from the group consisting of: GC373, GC375, GC376, GC523, GC543, GC546, GC551, GC554, GC583, GC587, GC591, GC597, GC772, GC774, and GC813.

15. The method of claim 13, wherein the GC molecule is GC376.

16. The method of claim 13, further comprising administering another therapeutic agent to said subject.

17. The method of claim 13, wherein the administration is an oral administration of the GC molecule.

18. The method of claim 17, wherein the oral administration is by a tablet comprising the antiviral composition.

19. The method of claim 13, wherein the administration is intravenous or subcutaneous administration.

20. The method of claim 13, wherein the subject is a human subject.

21. A GC 376 molecule having the molecular backbone shown in FIG. 1 and comprising a deuterated R₄ functional group or ester substitution at position R₂ structure shown in FIG. 2A.

22. A GC 373 molecule having the molecular backbone shown in FIG. 1 and comprising a deuterated R₃ functional group or ester substitution at position R₂ structure shown in FIG. 2B.
